(19) United States
(12) Patent Application Publication
(10) Pub. No.: US 2019/0133933 A1
(43) Pub. Date: May 9, 2019

(54) FORMULATIONS AND METHODS FOR REDUCTION OF INTRAOCULAR PRESSURE

(71) Applicant: CLEARSIDE BIOMEDICAL, INC., Alpharetta, GA (US)

(72) Inventors: Richard BECKMAN, Alpharetta, GA (US); Jesse YOO, Snellville, GA (US); Rafael Victor ANDINO, Grayson, GA (US); Glenn NORONHA, Atlanta, GA (US); Samirkumar PATEL, Atlanta, GA (US); Donna TARABORELLI, Alpharetta, GA (US); Vladimir ZARNITSYN, Atlanta, GA (US)

(21) Appl. No.: 16/097,303

(22) PCT Filed: May 1, 2017
(86) PCT No.: PCT/US2017/030439

§ 371 Date: Oct. 29, 2018

Related U.S. Application Data
(60) Provisional application No. 62/329,951, filed on Apr. 29, 2016.

(51) Int. Cl. A61K 9/00; A61K 47/36; A61F 9/00; A61F 9/007; A61M 37/00

(52) U.S. Cl. CPC A61K 9/0051; A61K 47/36; A61F 9/0017; A61F 9/00781; A61M 2250/00; A61M 2037/0023; A61M 2037/003; A61M 2037/0061; A61M 37/0015

(57) ABSTRACT

The current disclosure relates to formulations and methods for reducing intraocular pressure (IOP) in the eye of a subject in need thereof. The disclosure also relates to formulations and methods for reducing intraocular pressure (IOP) related to glaucoma in a subject. The methods provided include non-surgically administering a non-pharmacologically active injectable formulation to the eye of the subject by using an apparatus that is suitable for delivering the formulations. The methods provided also include placing a solid implant into the eye of the subject to create a controlled space in the suprachoroidal space (SCS) or the supraciliary space of the eye of the subject in need thereof. The present disclosure further comprises facilitating and improving the aqueous outflows in the eye through the trabecular meshwork outflow pathway and/or uveoscleral outflow pathway and thereby decreasing the intraocular pressure.

### CROSS-REFERENCE TO RELATED APPLICATIONS

This application claims priority to U.S. Provisional Application No. 62/329,951, filed Apr. 29, 2016, which is incorporated herein by reference in its entirety.

### FIELD OF THE INVENTION

The present disclosure relates to methods, formulations, and devices for reducing intraocular pressure (IOP) and for treating glaucoma. The methods provided include administration of non-pharmacologically active formulations and/or implants to the suprachoroidal space (SCS or supraciliary space of the eye).

### BACKGROUND OF THE INVENTION

Glaucoma is a multi-factorial, complex eye disease and a leading cause of visual impairment and blindness. Vision loss is associated with reduction of the visual field due to retinal ganglion cell degeneration and damage to the optic nerve. Three major types of primary glaucoma have been classified in humans. Primary Angle Open Glaucoma (POAG) is the most common type in most populations. The other classes of glaucoma are Primary Angle Closure Glaucoma (PACG) and Primary Congenital Glaucoma (PCG). Secondary glaucoma may be caused by injury, inflammation, certain drugs, and/or diseases and may also be of the open-angle or angle-closure type. Elevated intraocular pressure is a major risk factor for glaucoma and a common connection among all types of glaucoma. Reduction of intraocular pressure (IOP) has been shown to reduce the risk of vision loss in patients with glaucoma and is the mainstay of current therapy. Controlling intraocular pressure is a chronic problem that requires continuous management in order to reduce the risk of vision loss.

In addition to or in conjunction with glaucoma, causes of elevated IOP include inadequate drainage via the aqueous outflow pathways of the eye, certain medications, and eye trauma or other eye conditions.

Current management of IOP is commonly performed by pharmacological agents applied topically to the surface of the eye on a daily basis, laser surgery to increase intraocular aqueous outflow, or by surgical intervention or implantation of shunts or stents, all of which are associated with risks and/or insufficient efficacy. Currently available oculohypotensive drugs lower IOP by decreasing production of aqueous humor, increasing drainage of aqueous humor through the uveoscleral outflow pathway, or indirectly increasing outflow facility through the trabecular meshwork, or a combination of the above. There is a need in the art for improved methods of reducing IOP and for treating glaucoma and other diseases associated with elevated IOP.

### SUMMARY OF THE INVENTION

In one aspect, the present disclosure provides a method for reducing intraocular pressure (IOP) in a subject in need thereof. In some embodiments, the method comprises injecting a formulation or placing a solid implant into the suprachoroidal space (SCS) or the supraciliary space of the eye of the subject. In some embodiments, the formulation or implant is a non-pharmacologically active formulation or implant. In some embodiments, the injecting or placing forms a functional communication between the anterior chamber and the SCS of the eye. In another aspect, the present disclosure provides a method of treating glaucoma in a subject in need thereof. In some embodiments, the method for treating glaucoma in the subject comprises injecting a non-pharmacologically active formulation or placing a non-pharmacologically active solid implant into the suprachoroidal space (SCS) or the supraciliary space of the eye of the subject.

In some embodiments, the formulation in the present disclosure is a non-pharmacologically active injectable formulation which comprises a fluid injectate or a gel injectate. In some embodiments, the fluid injectate increases one or more of the aqueous outflow pathways of the eye. In some embodiments, the aqueous outflow pathways of the eye are the trabecular meshwork (TM) outflow pathway and/or the uveoscleral outflow pathway.

In some embodiments, the injectate in the present disclosure is water, an emulsion, or a hyaluronic acid based gel. In further embodiments, the formulation has a volume of from about 1 μl to about 1000 μl, about 10 μl to about 500 μl, or about 20 μl to about 200 μl. In another embodiment, the volume in the present disclosure is about 10 μl, about 20 μl, about 50 μl, about 100 μl, about 150 μl, about 200 μl, about 250 μl, about 300 μl, about 350 μl, about 400 μl, about 450 μl, or about 500 μl. In a further embodiment, the formulation has a volume of from about 10 μl to about 500 μl. In some embodiments, the formulation is hyaluronic acid. In further embodiments, the formulation is from about 0.5% to about 10% hyaluronic acid. In further embodiments, the formulation is 1% hyaluronic acid.

In some embodiments, the present disclosure provides that the intraocular pressure (IOP) is reduced for at least about 12 hours, at least about 24 hours, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, or at least about 6 months following injection of the formulation or placement of the solid implant. In some embodiments, the reduction in IOP is accomplished with or without expanding the SCS. In some embodiments, the method improves drainage of the canal or uvea and reduces fluid production by the ciliary body (CB).

In some embodiments, the present disclosure provides a method for increasing outflow through the trabecular meshwork (TM) outflow pathway and/or increases outflow through the uveoscleral outflow pathway. In some embodiments, the increased outflow is through both the TM and the uveoscleral outflow pathways. In further embodiments, the method increases outflow by flushing the system and/or affecting the ciliary body (CB), and/or by causing ocular tissues to become more porous.

In some embodiments, the present disclosure provides a method to cause mechanical deformation of the TM. In some embodiments, the administration of the non-pharmacologically active formulation or implant via administration to the SCS or supraciliary space of the eye creates an arc-shaped space in the SCS. In some embodiments, the arc-shaped space in the SCS disclosed herein comprises multiple injections of the formulation into the SCS. In other embodiments, the administration of the non-pharmacologically active formulation or implant to the SCS or supraciliary space of the eye of the subject, as provided herein, results in a non-mechanical change to the eye that reduces IOP. For example, in some embodiments, the methods cause a pharmacological response induced by administration of the non-pharmacologically active formulation or implant.

In some embodiments, the present disclosure provides an additional procedure for reducing IOP in the eye of the subject.

In some embodiments, the additional procedure is a surgery or the administration of a drug. In a further embodiment, the drug is administered to the subject via an ocular route of administration. In further embodiments, the drug is present in a pharmacologically active injectable formulation. In further embodiments, the drug is selected from the group consisting of cholinergic agents, latrunculins, ROCK inhibitors, prostaglandin analogues, α-adrenic receptor agonists, β-adrenergic receptor blockers, prostaglandin EP2 agonists, nitric oxid-donating prostaglandin F2α analogs, phosphylene iodide, and echothiopate iodide. In further embodiments, the drug is selected from the group consisting of Dorzolamide/Timolol (Cospot), Carteolol, Bimatoprost (Lumigan), Latanoprost (Xalatan), Brimonidine, Betaxolol (Betoptic), Travoprost, Dorzolamide (Trusopt), Timolol (Betimol), Pilocarpine, Brinzolamide (Azopt), Iopidine, Alphagan P, Betagan, OptiPranolol, Istalol, Timoptic-XE, Neptazane, Diamox Sequels, Isopto Carpine, Isopto Carbachol, Pilopine HS gel, Pilocarpine CL ophthalmic solution USP, Combigan, Simbrinza suspension, Travatan Z, Lumigan, Zioptan, and Xalatan. In further embodiments, the surgery is a minimally invasive glaucoma surgery (MIGS) and comprises the application of a shunt.

In some embodiments, the present disclosure provides a method and an additional procedure that act additively or synergistically to reduce IOP.

In some embodiments, the subject of the present disclosure is a mammal. In a further embodiment, the mammal is a human.

In one aspect, the present disclosure provides a method of treating glaucoma in a human in need thereof. In some embodiments, the method comprises non-surgically administering a non-pharmacologically active injectable formulation to the anterior suprachoroidal space (SCS) or the supraciliary space of the eye of the human by using a hollow microneedle. In some embodiments, upon administration, the intraocular pressure (IOP) is reduced by forming a functional communication between the anterior chamber and the SCS of the eye. The functional communication may be a mechanical and/or pharmacological response to the administration of the non-pharmacologically active formulation or implant to the SCS or supraciliary space of the eye.

### BRIEF DESCRIPTIONS OF THE FIGURES

FIG. 1 illustrates the two main exit pathways, trabecular meshwork pathway and uveosclearal pathway, for aqueous humor outflow.

FIG. 2 shows the current pharmacological treatments and investigational drugs targeting the trabecular meshwork outflow pathway, the uveoscleral outflow pathway, and the inflow pathway.

FIG. 3 shows the assignment of the canine normotensive model to various non-pharmacologically active injectable formulation groups. Group 1 represents the study control without injectate administration. Group 2 is administered with 100 μL emulsion or vehicle into the suprachoroidal space (SCS) of the eye. Group 3 is administered with 100 μL hyaluronic acid based gel into the suprachoroidal space (SCS) of the eye.

FIG. 4 illustrates an example of the timeline for administering the non-pharmacologically active injectable formulations.

FIG. 5 depicts an open cutaway illustration of an eye and an arc-shaped spacer formed via the methods disclosed herein. The anatomical locations illustrated are the globe, Schlemm's canal, limbus, pupil aperture, and the trabecular meshwork's Fontana's spaces. The “Arc spacer in SCS” is created by the injectable formulation. In some embodiments, multiple injections create the arc-shaped spacer. Abbreviations in the figure are TM: Trabecular Meshwork; SCS: Suprachoroidal Space

FIG. 6 shows the mean intraocular pressure for female beagle dogs administered a sham suprachoroidal injection to both eyes (Group 1). Each point represents mean±SD (n of 8 eyes).

FIG. 7 shows the IOP for female beagle dogs administered a sham suprachoroidal injection to both eyes (Group 1).

FIG. 8 shows the mean percent change in intraocular pressure for female beagle dogs administered a sham suprachoroidal injection to both eyes (Group 1). Each point represents mean±SD (n of 8 eyes).

FIG. 9 shows percent change in IOP for female beagle dogs administered a sham suprachoroidal injection to both eyes (Group 1).

FIG. 10 shows the mean intraocular pressure for female beagle dogs administered a suprachoroidal injection of Healon® OVD to the right eye and BSS to the left eye (Group 2). Each point represents mean±SD (n of 8 eyes).

FIG. 11 shows the mean percent change in intraocular pressure for female beagle dogs administered a suprachoroidal injection of Healon® OVD to the right eye and BSS to the left eye (Group 2). Each point represents mean±SD (n of 8 eyes).

FIG. 12 shows the mean delta percent change in intraocular pressure for female beagle dogs administered a suprachoroidal injection of Healon® OVD to the right eye and BSS to the left eye (Group 2). Each point represents mean±SD (n of 8 eyes).

FIG. 13 shows the mean intraocular pressure for female beagle dogs administered topical ocular latanoprost to the right eye and PBS to the left eye for poststudy efficacy challenge (Group 1). Each point represents mean±SD (n of 4 eyes).

FIG. 14 show the mean intraocular pressure for female beagle dogs administered topical ocular latanoprost to the right eye and PBS to the left eye for poststudy efficacy challenge (Group 2). Each point represents mean±SD (n of 8 eyes).

FIG. 15 shows the mean percent change in intraocular pressure for female beagle dogs administered topical ocular latanoprost to the right eye and PBS to the left eye for poststudy efficacy challenge (Group 1). Each point represents mean±SD (n of 4 eyes).

FIG. 16 shows the mean percent change in intraocular pressure for female beagle dogs administered topical ocular latanoprost to the right eye and PBS to the left eye for poststudy efficacy challenge (Group 2).

### DETAILED DESCRIPTION OF THE INVENTION

The present disclosure provides methods for reducing intraocular pressure (IOP) in the eye of a subject in need thereof. In some aspects, the present disclosure provides methods for the treatment of glaucoma, thereby addressing key needs in the fields of ocular therapeutics. The present disclosure provides methods to reduce intraocular pressure (IOP) in order to prevent vision loss, a problematic issue present in chronic glaucoma patients.

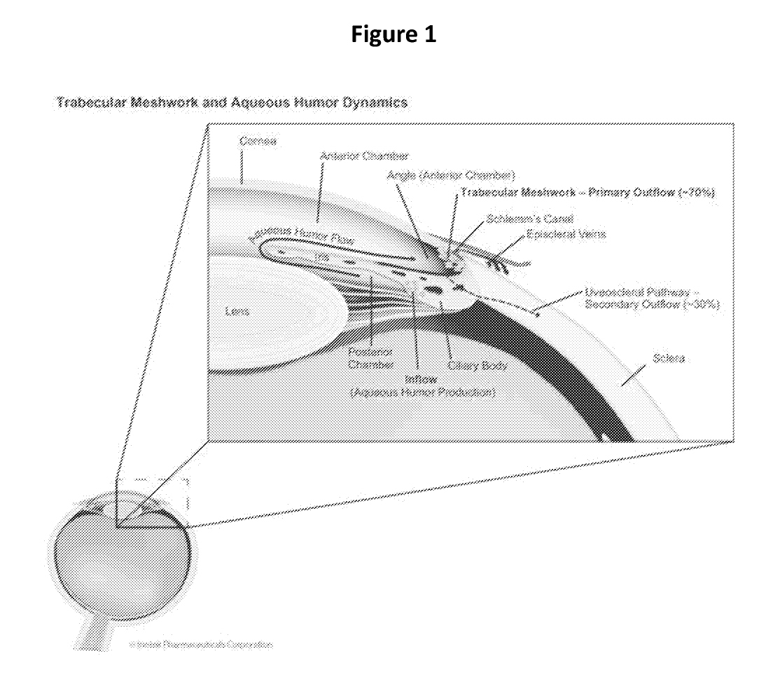

Intraocular pressure is most commonly managed via regulating the flow of aqueous humor fluid within the eye. This can be done by either reducing production of fluid, or making it easier for the fluid to flow out of the eye. In the case of facilitating outflow of aqueous humor, there are believed to be two outflow pathways. The majority of the fluid (70-90%) flows though the trabecular meshwork (primary or conventional outflow). The remainder (10-30%) of the fluid exits via the uveoscleral pathway (secondary or unconventional pathway). FIG. 1 is a schematic depiction of the pathways.

The term “suprachoroidal space” is used interchangeably herein with suprachoroidal, SCS, suprachoroid and suprachoroidia. The terms “SCS” and “supraciliary space” describe the potential space in the region of the eye disposed between the sclera and choroid. The region primarily is composed of closely packed layers of long pigmented processes derived from each of the two adjacent tissues; however, a space can develop in this region as a result of fluid or other material buildup in the suprachoroidal space and the adjacent tissues. In some embodiments of the present disclosure, a space or a disruption in the connective layers between the anterior chamber and the SCS is intentionally created by infusion of the formulations or implants provided herein.

In one aspect, the present disclosure provides methods for reducing intraocular pressure (IOP) comprising administering an injectate or placing a solid implant into the SCS or the supraciliary space of the eye of the subject. In some embodiments, the present disclosure provides methods for creating a functional communication between the anterior chamber and the SCS of the eye. In some embodiments, the functional communication between the anterior chamber and the SCS of the eye reduces the intraocular pressure (IOP). In some embodiments, the functional communication between the anterior chamber and the SCS of the eye reduces the IOP through mechanical, pharmacologic, or other means. Surprisingly, the reduction in IOP occurs with administration of the injectate or implate that does not include a pharmacologically active agent. For example, In some embodiments, the functional communication between the anterior chamber and the SCS of the eye allows aqueous flow to occur between the anterior chamber and the suprachoroidal space and therefore facilitates the accumulated fluids to flow out of the eye via both trabecular meshwork pathway and uveoscleral pathway. For example, in some embodiments, the methods provided herein may place tension in the trabecular meshwork (TM) and/or cause mechanical deformation of the TM by stretching on tissues such as the ciliary body (CB), ciliary muscle (CM), or other tissues. In some embodiments, the methods provided herein result in a reduction of fluid production as well as an increase in fluid outflow to reduce IOP in the eye. In some embodiments, the functional communication is provided via a controlled opening between the anterior chamber and the SCS of the eye. In some embodiments, the controlled opening or functional communication formed between the anterior chamber and the suprachoroidal space is similar to the cyclodialysis cleft which is a separation of the ciliary body from the scleral spur, creating a direct connection between the anterior chamber and the suprachoroidal space. In other embodiments, the functional communication between the anterior chamber and the SCS may be a pharmacological response to the methods provided herein. In some embodiments, the functional communication between the anterior chamber and the SCS results in a reduction in IOP that is a result of one or both of a mechanical or a pharmacological effect.

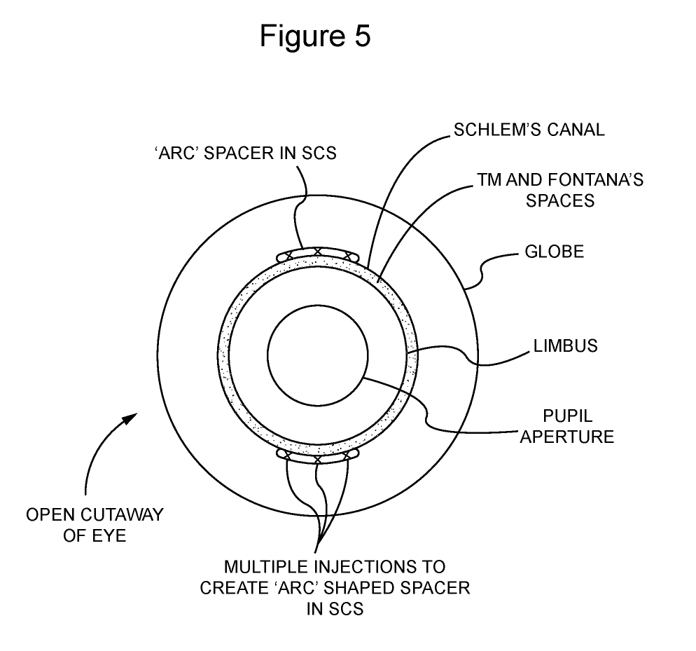

In some embodiments, the functional communication between the anterior chamber and the SCS of the eye is provided via an arc-shaped spacer in the SCS (FIG. 5). In further embodiments, the arc-shaped spacer is created by multiple injections according to the methods provided herein. In further embodiments, the arc-shaped spacer places tension in the TM and leads to both a decrease in fluid production and an increase in the fluid outflow of the eye.

In some embodiments, the methods provided herein reduce the IOP by creating a space in the SCS, disrupting the connective layers between the anterior chamber and the SCS, improving drainage of the canal or uvea, mechanically deforming the TM, causing compression on the globe, causing inflammation, reducing fluid production by the ciliary body (CB), and/or increasing outflow through the aqueous outflow pathways of the eye. In some embodiments, the outflow is increased by, for example, flushing fluid through the outflow pathways, causing the tissues of the eye to become more porous, and/or having an effect on the CB that improves outflow. In some embodiments, the methods provided herein result in local inflammation, which in turn reduces the IOP.

In one aspect, the methods provided herein comprise administration of an injectate or placement of an implant in the eye of a subject. In further embodiments, administration of the injectate is by injection into the SCS. The injecate or implant in some embodiments does not include a pharmacologically active agent. Thus, in some embodiments, the present disclosure provides methods for reducing IOP and/or treating glaucoma comprising administration of a solid injectate, gel, or hyaluronic acid into the SCS, wherein the solid injectate, gel, or hyaluronic acid does not include a pharmacological agent. For example, in some embodiments, the solid injectate, gel, or hyaluronic acid is injected into the suprachoroidal space via a microneedle.

Without wishing to be bound by theory, in some embodiments, the methods provided herein comprise administration of an injectate or placement of an implant that causes a shift in the lens/iris diaphragm. In further embodiments, the shift in the lens-iris diaphragm results in an increase in trabecular meshwork outflow. In other embodiments, the injectate or implant causes a functional detachment of the ciliary body, leading to decreased aqueous formation. In some embodiments, the detachment of the ciliary body is partial detachment. In some embodiments, the injectate or implant causes a pull on the trabecular meshwork. In further embodiments, the pull on the trabecular meshwork causes distention and upregulation of matrix metalloproteinases leading to increased trabecular meshwork outflow. In some embodiments, the injectate or implant causes a mechanical and/or functional change in the iris and/or ciliary body. In further embodiments, the mechanical and/or functional change in the iris and/or ciliary body results in increased uveoscleral outflow.

The reduction of intraocular pressure (IOP) described herein can be achieved either with or without expanding the space between the anterior chamber and the suprachoroid. Without wishing to be bound by theory, it is thought that, in some embodiments, the mechanism by which the expansion can occur is because the injection of the fluid causes a disruption in the connective layers between the anterior chamber and the suprachoroidal space. This allows aqueous flow to occur between the anterior chamber and the aqueous exit pathways. Even though the fluid is injected into the anterior suprachoroidal space or supraciliary space, the disruption could impact both exit pathways (trabecular meshwork and uveoscleral). In addition, the magnitude and duration of effect could be tuned depending on the formulation, volume of the formulation, rate of injection, location of injection, etc.

Thus, in some embodiments, the methods provided herein comprising administration of a fluid or gel formulation or the administration or placement of an implant can be carried out with any fluid or gel formulation or implant, i.e., the formulation or implant does not comprise an active agent. Surprisingly, the administration of the non-pharmacologically active fluid or gel injectate or solid implant results in a reduction in IOP. The reduction in IOP is surprising, in part, because administration of any fluid or solid into the eye would not be expected to reduce IOP in the eye. In fact, administration of a fluid or implant other than an IOP-reducing agent such as prostaglandins would be expected to either not affect or to increase, rather than decrease, pressure in the closed system of the eye. The present disclosure provides the unexpected finding that administration of the non-pharmacologically formulation or solid implant by injection into the SCS or supraciliary space of the eye reduces intraocular pressure in the absence of a drug agent.

In some embodiments, the injectate comprises a fluid or a gel-based formulation. The fluid or gel injectate may be, for example, water, a buffer, an emulsion, or a gel. The gel may be any gel known in the art, for example, hyaluronic acid based gel or hydrogel. In some embodiments, the fluid or gel injectate is hyaluronic acid or hyaluronan. Hyaluronic acid is an anionic, non-sulfated glycosmaminoglycan. In further embodiments, the hyaluronic acid is from about 0.1% to about 50% hyaluronic acid. For example, in some embodiments, the hyaluronic acid is 0.1%, 0.5%, 1%, 2%, 3%, 4%, 5%, 10%, 15%, 20%, 30%, 40%, or 50% hyaluronic acid. In certain embodiments, the hyaluronic acid is 1% hyaluronic acid. For example, in some embodiments, the hyaluronic acid is Healon® OVD (1% hyaluronic acid). In some embodiments, the implant is a solid implant.

The normal IOP in a human subject is about 12-22 mm Hg. In some embodiments, the present disclosure provides methods for reducing IOP in a subject in need thereof comprising injecting a formulation or placing an implant into the SCS or supraciliary space of the eye of the subject, wherein the IOP is reduced by about 1%, about 5%, about 6%, about 7%, about 7%, about 9%, about 10%, about 15%, about 20%, about 25%, about 30%, about 35%, or more relative to the pre-treatment level. In some embodiments, the IOP is reduced by about 1%, about 5%, about 6%, about 7%, about 7%, about 9%, about 10%, about 15%, about 20%, about 25%, about 30%, about 35%, or more relative to a control treatment. In some embodiments, the reduction in IOP is sustained for a period of time after administration of the formulation or implant. For example, in some embodiments, the reduction in IOP is sustained for at least 1, 2, 3, 4, 5, 6, 7, 8, 9, 10, 11, 12, 13, or 14 days. In some embodiments, the reduction in IOP is sustained for at least 2, 3, 4, 5, 6, 7, 8, 9, 10, 11, or 12 weeks. In some embodiments, the reduction in IOP is sustained for at least 3, 4, 5, 6, 7, 8, 9, 10, 11, or 12 months. In some embodiments, the reduction in IOP is sustained for a period of longer than a year.

In one aspect, the present disclosure provides methods for reducing IOP and/or treating glaucoma in a subject in need thereof comprising injecting a formulation or placing an implant into the SCS or supraciliary space of the eye of the subject, wherein the method further comprises one or more additional procedure for reducing the IOP in the subject. For example, in some embodiments, the methods comprise a combination of an injection of a formulation or placing of an implant into the SCS or supraciliary space of the eye as provided herein, and an additional approach such as a surgical procedure, implantation of a shunt or stent, or administration of a drug formulation. In some embodiments, the combination of the novel methods provided herein with one or more additional procedures act additively or synergistically to reduce the IOP; and/or the combination of the novel methods provided herein with one or more additional procedures act additively or synergistically to treat glaucoma in a subject in need thereof. In some embodiments, the term “additive” as used herein means that the effect of the two or more methods (e.g., administration of the non-pharmacologically active formulation or implant to the SCS or supraciliary space of the eye as provided herein, and the one or more additional procedure) provides an effect in reducing IOP and/or treating glaucoma that is greater than the effect provided by one of the methods/procedures alone. As used herein, the term “synergistically” refers to a situation in which the effect of the two procedures together is greater than the sum of the individual effects of each procedure when carried out alone.

For example, in some embodiments, the combination of the methods for reducing IOP or treating glaucoma provided herein act additively or synergistically with one or more known IOP-reduction procedures or glaucoma treatments. Known IOP-reduction procedures and glaucoma treatments include surgical procedures and drug administration. Surgical procedures include, for example, Microinvasive Glaucoma Surgery (MIGS), which involves using a device that facilitates drainage of fluid through an outflow pathway. Through this surgery, a channel is created that allows easier movement of aqueous through the outflow pathway of choice. For example, implantations of shunts or stents (e.g., iStent, Tarbectome, Hydrus Microstent, XEN glaucoma implant, and CyPass Micro-Stent) use this approach. Other surgical procedures used to attempt to reduce IOP and/or treat glaucoma include laser trabeculoplasty, trabeculectomy, iridotomoy, iridectomy, sclerectomy, or viscocanalostomy.

In some embodiments, the known IOP reduction procedure and/or glaucoma treatment is administration of one or more drugs selected from the group consisting of prostaglandins, cholinergic agents, latrunculins, ROCK inhibitors, prostaglandin analogues, α-adrenic receptor agonists, β-adrenergic receptor blockers, prostaglandin EP2 agonists, nitric oxide-donating prostaglandin F2α analogs, phosphylene iodide, and echothiopate iodide. In some embodiments, the known IOP reduction procedure and/or glaucoma treatment is administration of one or more drugs selected from the group consisting of Dorzolamide/Timolol (Cospot), Carteolol, Bimatoprost (Lumigan), Latanoprost (Xalatan), Brimonidine, Betaxolol (Betoptic), Travoprost, Dorzolamide (Trusopt), Timolol (Betimol), Pilocarpine, Brinzolamide (Azopt), Iopidine, Alphagan P, Betagan, OptiPranolol, Istalol, Timoptic-XE, Neptazane, Diamox Sequels, Isopto Carpine, Isopto Carbachol, Pilopine HS gel, Pilocarpine CL ophthalmic solution USP, Combigan, Simbrinza suspension, Travatan Z, Lumigan, Zioptan, and Xalatan.

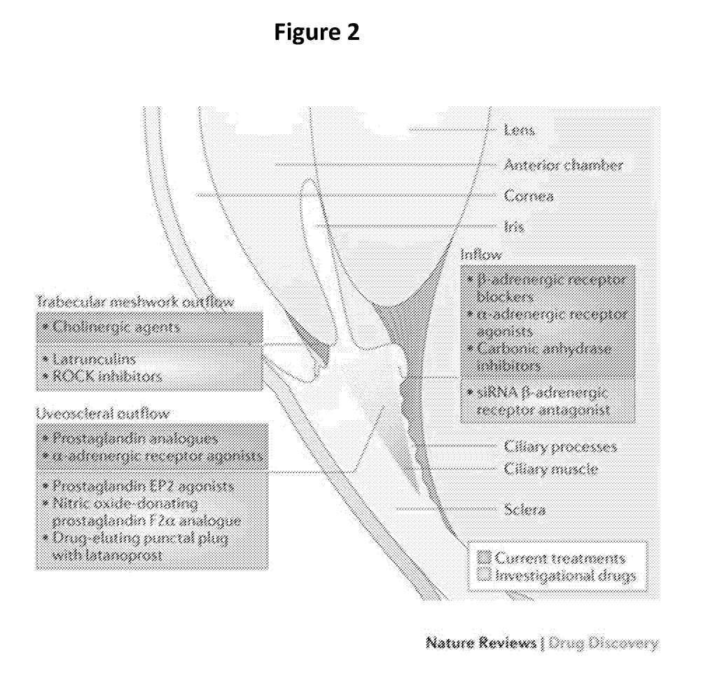

Prostaglandins are currently the most commonly prescribed medication for glaucoma. Many of the current pharmacological treatments target the uveoscleral pathway. This pathway drains fluid through the suprachoroidal space (SCS) and eventually outside the eye through the sclera. In general, as summarized in FIG. 2, cholinergic agents, latrunculins, and ROCK inhibitors are commonly used for trabecular meshwork outflow. Prostaglandin analogues, α-adrenergic receptor agonists, prostaglandin EP2 agonists, nitric oxide-donating prostaglandin F2α analogue, and drug-eluting punctal plug with latanoprost are commonly used for uveoscleral outflow pathway.

In some embodiments, the intraocular pressure reduction effect provided by the methods disclosed herein remains at least as long as the injectate or implant is present in the space. In another embodiment, the intraocular pressure is reduced for a or at least about 12 hours, at least about 24 hours, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, at least about 6 months, or longer following injection of the formulation.

In another embodiment, intraocular pressure, pupil diameter, and other ophthalmic exams are regularly measured post-administration, for example, 1 day, 2 days, 3 days, 5, days, 7 days, 1 week, 2 weeks, 3 weeks, 4 weeks, 5 weeks, 6 weeks, 7 weeks, 8 weeks, 9 weeks, 10 weeks, 11 weeks, 12 weeks, or longer post-administration, and all values in between.

In one embodiment, while the reduction in IOP via the methods described in the present disclosure can be accomplished without the use of a pharmacological agent, alternatively, a pharmacologically active injectable formulation can be administered before, after, or simultaneously with the administration of the non-pharmacologically active injectable formulation to the eye of the subject by using the non-surgical methodology as described herein.

The “pharmacologically active injectable formulation” refers to any drug that can treat or alleviate the symptoms of glaucoma. Examples include but are not limited to Dorzolamide/Timolol (Cospot), Carteolol, Bimatoprost (Lumigan), Latanoprost (Xalatan), Brimonidine, Betaxolol (Betoptic), Travoprost, Dorzolamide (Trusopt), Tim olol (Betimol), Pilocarpine, Brinzolamide (Azopt), Iopidine, Alphagan P, Betagan, OptiPranolol, Istalol, Timoptic-XE, Neptazane, Diamox Sequels, Isopto Carpine, Isopto Carbachol, Pilopine HS gel, Pilocarpine CL ophthalmic solution USP, Combigan, Simbrinza suspension, Travatan Z, Lumigan, Zioptan, or Xalatan.

The drug can also be selected from any of the following types of treatments: cholinergic agents, latrunculins, and ROCK inhibitors, prostaglandin analogues, α-adrenergic receptor agonists, prostaglandin agonists, nitric oxide-donating prostaglandin F2α analogue, and drug-eluting punctal plug with latanoprost, beta-adrenergic receptor blockers, α-adrenergic receptor agonists, carbonic anhydrase inhibitors, siRNA beta-adrenergic receptor antagonists, phosphylene iodide, and echothiopate iodide.

In one embodiment, the amount of the non-pharmacologically active injectable formulation or of the pharmacologically active injectable formulation is from about 1 μL to about 1000 μL, e.g., from about 10 μL to about 200 μL, or from about 50 μL to about 150 μL.

In one embodiment, the present disclosure comprises a method to treat a human in need of treatment of glaucoma by using a hollow microneedle to non-surgically administer a non-pharmacologically active injectable formulation to the SCS or supraciliary space of one or both of the eyes of the human. In another embodiment, at least one optional pharmacologically active injectable formulation can be non-surgically administered to the SCS or supraciliary space of one or both of the eyes of the human by using a hollow microneedle. In another embodiment, the optional pharmacologically active formulation can be administered before, after, or simultaneous with the administration of the non-pharmacologically active injectable formulation to the eye of the human. In another embodiment, a functional communication is formed between the anterior chamber and the suprachoroidal space (SCS) of the eye upon the administration of non-pharmacologically active formulation and results in the reduction of the intraocular pressure (IOP).

As used herein, the terms “about” generally mean plus or minus 20% of the value stated. For example, about 1.0 would include 0.8 to 1.2; about 10 would include 1 to 12, about 1000 would include 800 to 1200.

In one embodiment, the administration results in delivering the formulation to the SCS or supraciliary space of one or both eyes of the subject. “Non-surgical administration” and related terms refer to using methods that do not require general anesthesia and/or retrobulbar anesthesia (also referred to as a retrobulbar block), therefore is minimally invasive and safe. “Non-pharmacologically” is defined as a characteristic of a substance in which its composition does not exert a medicinal or therapeutic effect on the cell, tissue, organ, or organism. “Eye” or “ocular tissue” includes both the anterior segment of the eye (i.e., the portion of the eye in front of the lens) and the posterior segment of the eye (i.e., the portion of the eye behind the lens). In one embodiment, “injectable” refers to any fluid substance that is capable of being injected and can be delivered by an apparatus capable of contacting the eye. In another embodiment, the apparatus can include, but is not limited to a hollow microneedle.

As used herein, “hollow microneedle” or “microneedle” refers to a conduit body having a base, a shaft, and a tip end suitable for insertion into the sclera and other ocular tissue and has dimensions suitable for minimally invasive insertion and drug formulation infusion as described herein. That is, the microneedle has a length or effective length that does not exceed about 2000 microns and a width (or diameter) that does not exceed about 600 microns.

As used herein, the terms “hollow” includes a single, straight bore through the center of the microneedle, as well as multiple bores, bores that follow complex paths through the microneedles, multiple entry and exit points from the bore(s), and intersecting or networks of bores. That is, a hollow microneedle has a structure that includes one or more continuous pathways from the base of the microneedle to an exit point (opening) in the shaft and/or tip portion of the microneedle distal to the base.

Both the “length” and “effective length” of the microneedle encompass the length of the shaft of the microneedle and the bevel height of the microneedle. In some embodiments, the microneedle used to carry out the methods described herein comprises one of the devices disclosed in International Patent Application Publication No. WO2014/179698 (Application No. PCT/US2014/036590), filed May 2, 2014 and entitled “Apparatus and Method for Ocular Injection,” incorporated by reference herein in its entirety for all purposes. In some embodiments, the microneedle used to carry out the methods described herein comprises one of the devices disclosed in International Patent Application Publication No. WO2014/036009 (Application No. PCT/US2013/056863), filed Aug. 27, 2013 and entitled “Apparatus and Method for Drug Delivery Using Microneedles,” incorporated by reference herein in its entirety for all purposes. In some embodiments, the microneedle used to carry out the methods described herein comprises one of the devices disclosed in US Patent Application Publication No. 2015/0038905, filed May 2, 2014 and entitled “Apparatus and Methods for Ocular Injection,” incorporated by reference herein in its entirety for all purposes

In various embodiments, the microneedle may have a length of about 50 microns to 2000 microns. In another particular embodiment, the microneedle may have a length of about 150 microns to about 1500 microns, about 300 microns to about 1250 microns, about 500 microns to about 1250 microns, about 700 microns to about 1000 microns, or about 800 to about 1000 microns. In a preferred embodiment, the length of the microneedle is about 1000 microns. In various embodiment, the proximal portion of the microneedle has a maximal width or cross-sectional dimension of about 50 microns to 500 microns, about 50 microns to about 400 microns, about 100 microns to about 400 microns, about 200 microns to about 400 microns, or about 100 microns to about 250 microns, with an aperture diameter of about 5 microns to about 400 microns. In a particular embodiment, the proximal portion of the microneedle has a maximal width or cross-sectional dimension of about 400 microns. Those skilled in the art will appreciate, however, that in embodiments in which the tip of the microneedle is beveled that the aperture diameter may be greater than the outer diameter of the proximal portion of the microneedle. The microneedle may be fabricated to have an aspect ratio (width:length) of about 1:1.5 to about 1:10.

The microneedle can have a straight or tapered shaft. In one embodiment, the diameter of the microneedle is greatest at the base end of the microneedle and tapers to a point at the end distal the base. The microneedle can also be fabricated to have a shaft that includes both a straight (i.e., untapered) portion and a tapered (e.g., beveled) portion. The microneedles can be formed with shafts that have a circular cross-section in the perpendicular, or the cross-section can be non-circular. The tip portion of the microneedles can have a variety of configurations. The tip of the microneedle can be symmetrical or asymmetrical about the longitudinal axis of the shaft. The tips may be beveled, tapered, squared-off, or rounded. In particular embodiments, the microneedle may be designed such that the tip portion of the microneedle is substantially the only portion of the microneedle inserted into the ocular tissue (i.e., the tip portion is greater than 75% of the total length of the microneedle, greater than 85% of the total length of the microneedle, or greater than about 95% of the total length of the microneedle). In other particular embodiments, the microneedle may be designed such that the tip portion is only a portion of the microneedle that is inserted into the ocular tissue and generally has a length that is less than about 75% of the total length of the microneedle, less than about 50% of the total length of the microneedle, or less than about 25% of the total length of the microneedle. For example, in one embodiment the microneedle has a total effective length between 500 microns and 1000 microns, wherein the tip portion has a length that is less than about 400 microns, less than about 300 microns, or less than about 200 microns.

As used herein, the words “proximal” and “distal” refer to the direction closer to and away from, respectively, an operator who would insert the medical device into the patient, with the tip-end (i.e., distal end) of the device inserted inside a patient's body first. Thus, for example, the end of a microneedle described herein first inserted inside the patient's body would be the distal end, while the opposite end of the microneedle (e.g. the end of the medical device being manipulated by the operator) would be the proximal end of the microneedle.

The microneedle and/or any of the components included in the embodiments described herein is/are formed and/or constructed of any suitable biocompatible material or combination of materials, including metals, glasses, semi-conductor materials, ceramics, or polymers. Examples of suitable metals include pharmaceutical grade stainless steel, gold, titanium, nickel, iron, gold, tin, chromium, copper, and alloys thereof. The polymer can be biodegradable or non-biodegradable. Examples of suitable biocompatible, biodegradable polymers include polylactides, polyglycolides, polylactide-co-glycolides (PLGA), polyanhydrides, polyorthoesters, polyetheresters, polycaprolactones, polyesteramides, poly (butyric acid), poly (valeric acid), polyurethanes and copolymers and blends thereof. Representative non-biodegradable polymers include various thermoplastics or other polymeric structural materials known in the fabrication of medical devices. Examples include nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly (vinyl imidazole), chlorosulphonate polyolefins, polyethylene oxide, blends and copolymers thereof. Biodegradable microneedles can provide an increased level of safety compared to non-biodegradable ones, such that they are essentially harmless even if inadvertently broken off into the ocular tissue.

The microneedle device may comprise a means for controllably inserting, and optionally retracting, the microneedle into the ocular tissue. In addition, the microneedle device may include means of controlling the angle at which the at least one microneedle is inserted into the ocular tissue (e.g., by inserting the at least one microneedle into the surface of the ocular tissue at an angle of about 90 degrees).

In one embodiment, the “subject” refers to humans and any non-human mammals. The definition of non-human mammals is well known in the art. Additionally, the selected non-human mammals are suitable for the method described in the present disclosure.

The following examples are offered by way of illustration and not by way of limitation.

### Example 1

Examining the Effects of Fluid Injection into the Suprachoroidal Space (SCS)

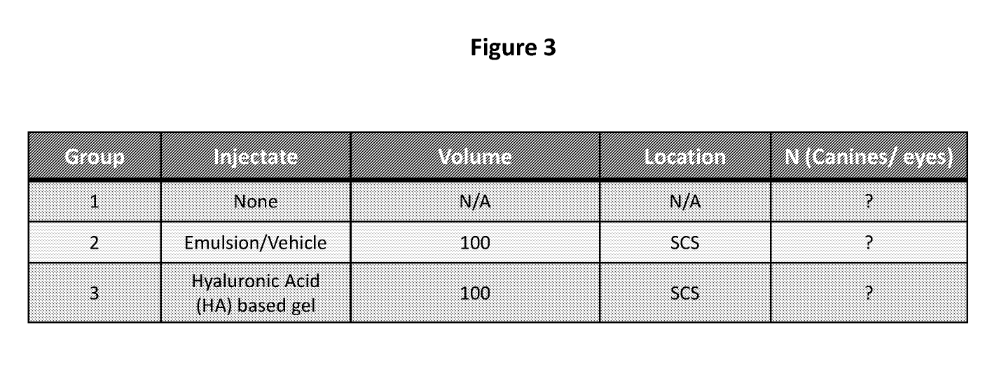

In order to determine the effects of administering the non-pharmacologically active injectable formulation to the anterior suprachoroidal space (SCS) of the eye, a canine normotensive model will be used. In brief, canines will be randomly assigned to three groups with three distinct injectate treatments (FIG. 3). Eyes will be examined. Group 1 will serve as the control and no injectate will be provided. Group 2 will be treated with 100 μl emulsion/vehicle which will be injected via microneedle into the suprachoroidal space (SCS). Group 3 will be treated with 100 μl hyaluronic acid based gel which will also be injected at the suprachoroidal space (SCS). All experimental procedures will comply with the protocols approved by The Institutional Animal Care and Use Committee (IACUC).

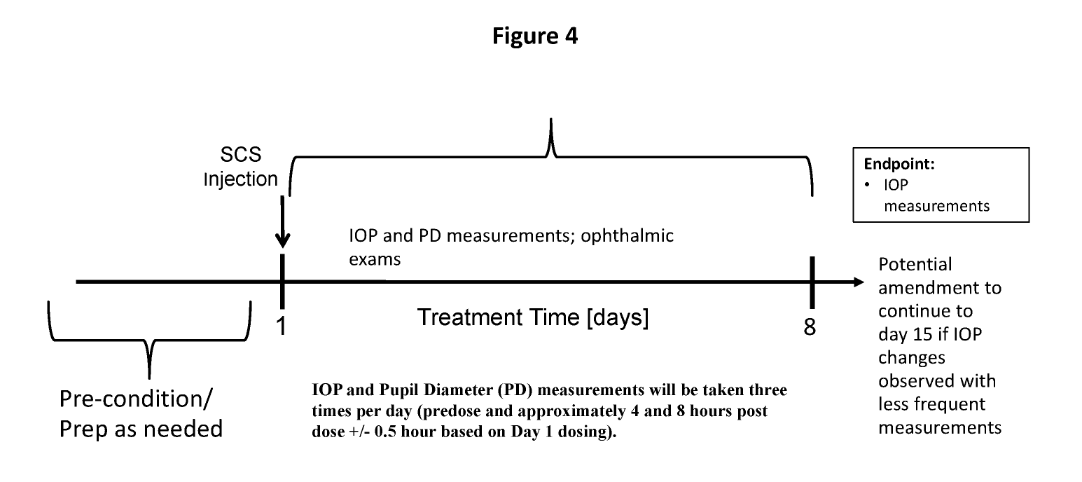

After the pre-condition stage, all canines in Group 2 and Group 3 will be injected with the first administration of their assigned injectates. Intraocular Pressure (IOP) measurements, pupil diameter (PD) measurements, or any other ophthalmic exams will be taken three times per day on day 1, 2, 3, 5, and 7 throughout the 8-day experiment (FIG. 4). Tonopen, Goldmann Applanation Tonometer, or TonoVet Tonometer will be used to measure the intraocular pressure (IOP). All instruments used to measure IOP will be calibrated according to the manufacturer's specifications and documents. The three time points will be before the administration of the injectate, and 4 hours and 8 hours (+/−0.5 hours) post-administration. Alternatively, the course of the experiments will be extended to 15 days if IOP changes are observed with less frequent measurements.

### Example 2

The Effects of Hyaluronic Acid on the Intraocular Pressure (IOP) after the Injection into the Suprachoroidal Space (SCS)

To assess the IOP lowering effect of hyaluronic acid, a single suprachoroidal injection of Healon® OVD (1% hyaluronic acid) was administered in normotensive dogs. Twelve female beagle dogs were assigned to two treatment groups as shown in Table 1. In brief, before the assignment of the treatment groups, the animals were first trained for IOP measurements. Overall health and predose ophthalmic examination were also conducted. Based on overall acclimation to the pharmacodynamic measurements, the animals were selected for Group 1 (n=4) and Group 2 (n=8). Animals in Group 1 received a sham suprachoroidal needle insertion to each eye with no formulation administered, whereas animals in Group 2 received a suprachoroidal administration of Healon® OVD to the right eye and balanced salt solution (BSS; or vehicle) to the left eye.

TABLE 1
Study Design

| | | | | Target | | |
|---|---|---|---|---|---|---|
| | | | | Dose | | Target |
| | | | | Level | | Dose |
| | Number of | Dose Route | | (μg/eye) | | Volume |
| Group | Female Animlas | OD | OS | OD | OS | (μg/eye) |
| 1 | 4 | Suprachoroidal^{a} | Suprachoroidal^{a} | 0 | 0 | 0 |
| 2 | 8 | Suprachoroidal^{b} | Suprachoroidal^{c} | 100 | 0 | 100 |

OD: Right eye;
OS: Left eye;
^{a}Animals served as the non-dosed control and received a sham needle insertion;
^{b}Eye was dosed with Healon ® OVD;
^{c}Eye was dosed with vehicle.

Before performing the sham needle insertion or administration, animals were anesthetized with butorphanol, dexmedetomidine, midazolam, and glycopyrrolate. Animals were also administered carprofen at least 30 minutes prior to sedation, and carprofen, tramadol, and bland ophthalmic ointment at least 6 hours post-dose. Other analgesic agents such as neomycin, polymyxin B sulfates drops or ointment, tramadol, NSAIDs, meloxicam, and buprenorphine can also be used. Following application of topical anesthetic, eyes were rinsed with an iodine solution for approximately 2 minutes followed by a saline rinse. Detailed observations, with particular attention paid to the eyes, were made pre-dose and post-dose on Study Day 1. A board-certified veterinary ophthalmologist conducted ophthalmic examination pre-dose and on Study Days 2 and 8. A slitlamp biomicroscope was used to examine the adnexa and anterior portion of each eye. In addition, the eyes were dilated with mydriatic agent and the ocular fundus of each eye was examined using an indirect ophthalmoscope.

Pharmacodynamic readings of IOP were collected at various time points before, during, and after the dosing procedures. IOP was measured for all available animals at least three times per week, once per day for at least 2 weeks prior to the day of dose administration. For example, the IOP measurement for Group 1 was scheduled as the following: −1 (pre-anesthesia), 0 (pre-sham injection), 0.5, 1, 2, 4, 8, and 24 hours post-sham injection. The IOP measurement for Group 2 was scheduled as the following: −1 (pre-anesthesia), 0 (pre-dose), 0.5, 1, 2, 4, 8, 24, 28, 32, 48, 52, 56, 144, 148, 152, 192, 196, 200, 288, 292, 296, 336, 340, and 344 hours post-dose (±0.5 hours after 8 hours).

IOP was measured by using a TonoVet according to a study-specific procedure. IOP measurements consisted of three independent readings per eye per time point from each eye. Following approximately 4 weeks of washout, animals in both groups were administered latanoprost in the right eye and phosphate-buffered saline (PBS) in the left eye, and IOP measurements were followed for 24 hours. As applicable, the dosing solution was drawn up into a 1-mL luer-lock syringe using a standard 21-gauge, 1-inch needle. Bubbles were expressed and the standard needle was replaced by a 30-gauge microneedle 1100 μm in length. A single suprachoroidal injection of 100 μL given over 5 to 10 seconds was administered to each eye (5 to 6 mm from the limbus, in the superior temporal quadrant). Following the injection, the needle was kept in the eye for approximately 10 seconds before being withdrawn. Upon withdrawal of the microneedle, a cotton-tipped applicator (CTA, dose wipe) was placed over the injection site for approximately 10 seconds. The right eye was dosed first and all post-dose times were based on the time of dosing of the second (left) eye.

Approximately 8 weeks after dose administration, all animals underwent a poststudy efficacy challenge. A positive displacement pipette was used to administer a single 35-μL dose of latanoprost to the right eye and phosphate-buffered saline (PBS) to the left eye. IOP was measured prior to dose administration (approximately −1 and 0 hours), and at 2, 4, 6, 8, and 24 hours following treatment. Statistical analyses such as 2-way ANOVA and Student-Newman-Keuls tests were also used for analyzing the IOP data.

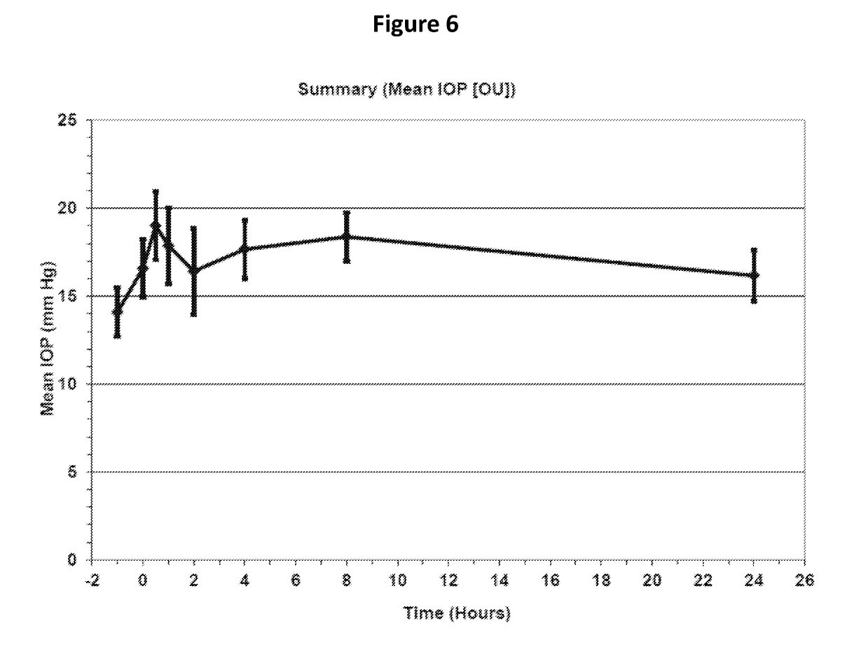

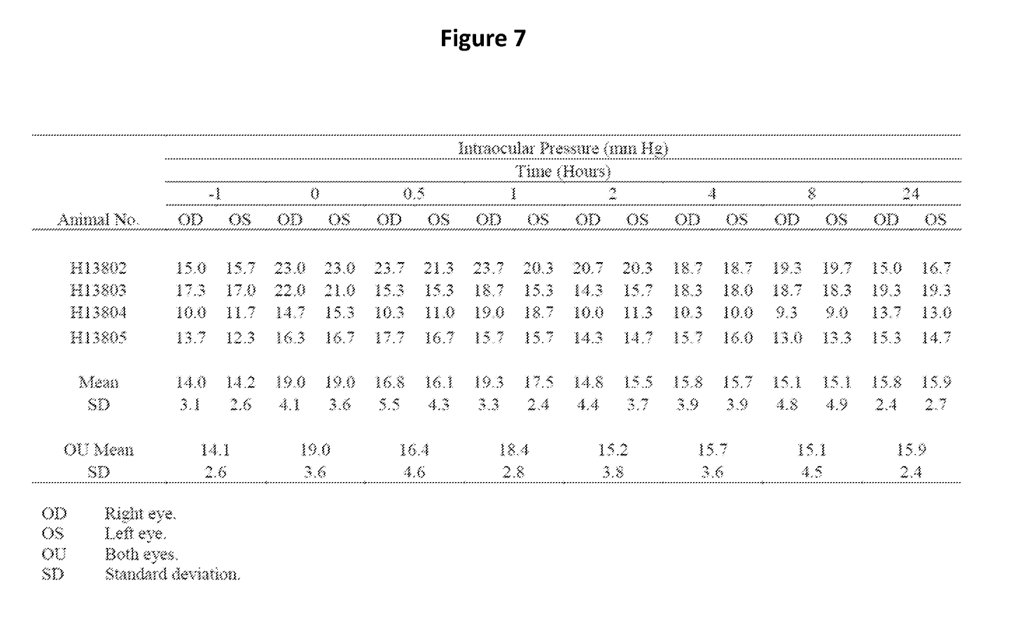

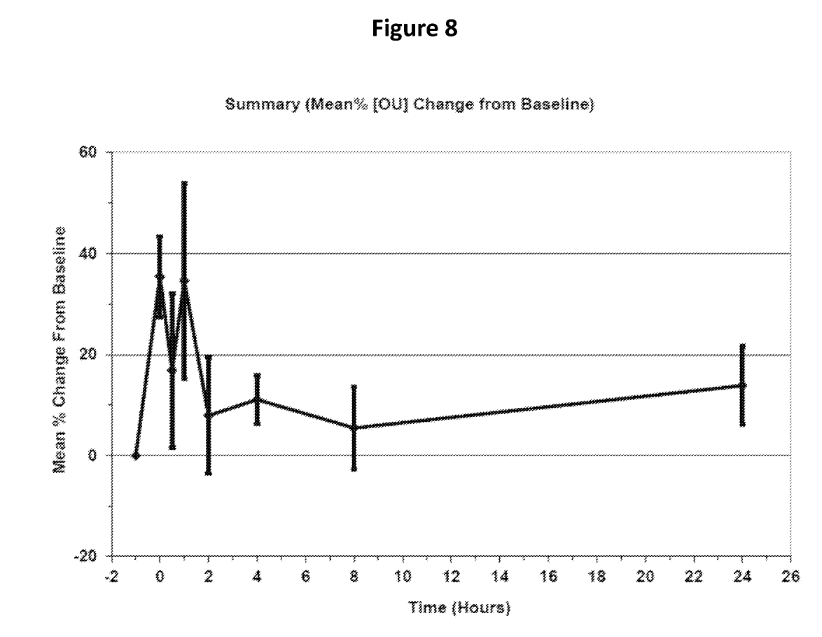

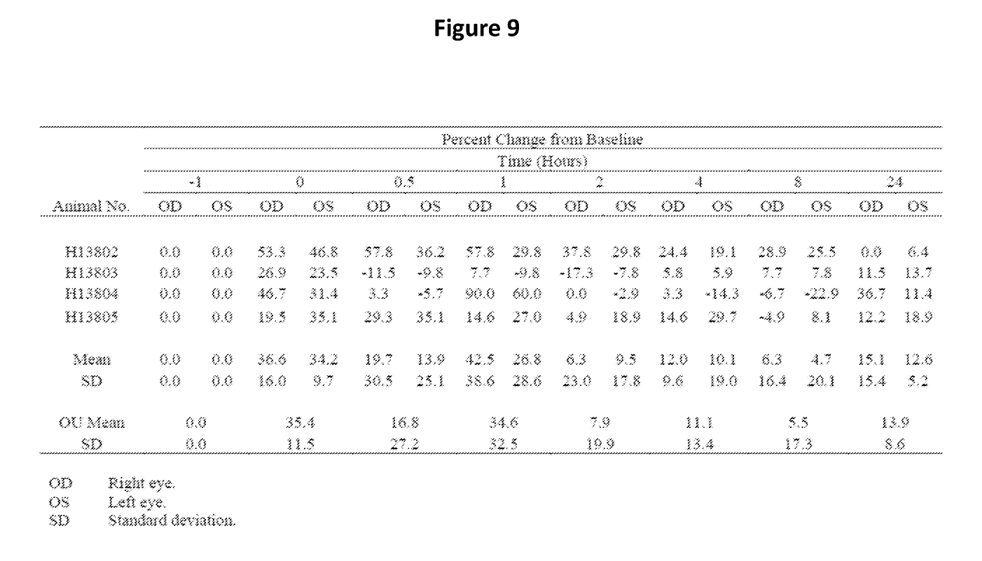

The mean IOP values for Group 1 (sham controls) increased similarly in right and left eyes, and then decreased towards baseline following recovery from anesthesia (FIG. 6 and FIG. 7). As shown in FIG. 6, the IOP increased up to close to 40% one hour after the sham operation and decreased to about 15% by 24 hours in both eyes. The mean IOP values in Group 2 exhibited a similar response in the early time points during anesthesia (FIG. 8 and FIG. 9). These data may suggest that the procedure/anesthesia leads to an increase in mean IOP values, possibly due to medicants administered and the positioning of the animals during measurements. The mean IOP values in Group 1 remained slightly elevated through 24 hours post-dose, albeit with increased variability.

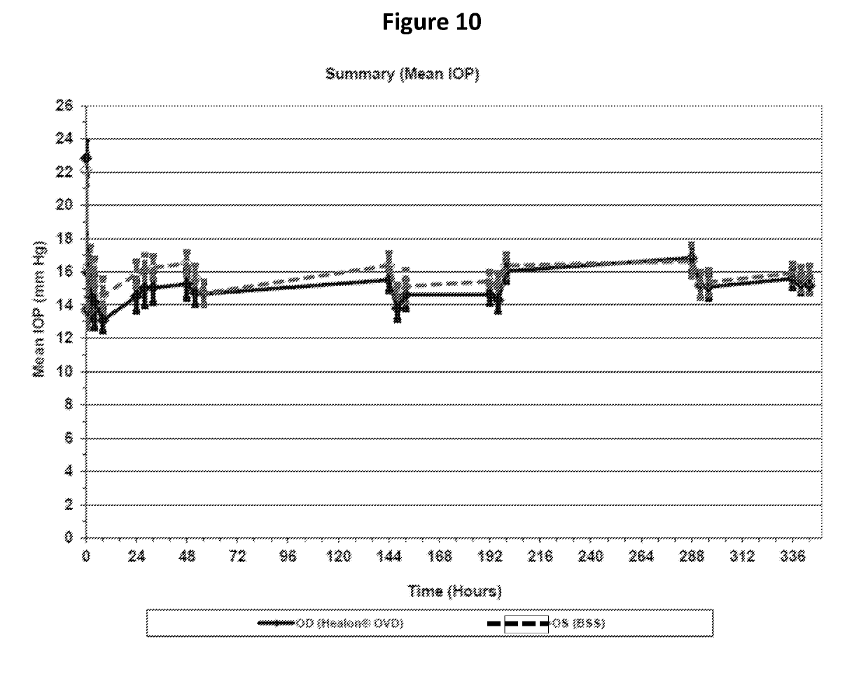

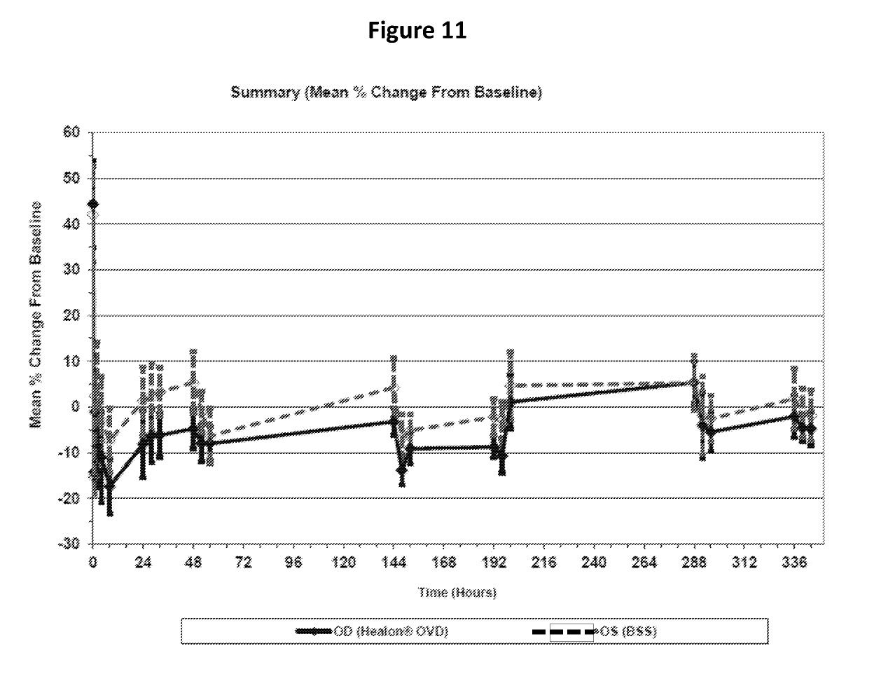

Following the IOP readings during anesthesia for Group 2 animals, either BSS (OS) or Healon® (OD) were injected into the suprachoroidal space (SCS). The IOP was monitored at selected intervals through 344 hours post-dose. An injection of BSS into the SCS had minimal effect on the mean IOP values (FIG. 10), and the pattern appeared similar to what would be expected for diurnal variation. The magnitude of the inflection through 144 hours post-dose was somewhat greater than expected. The same pattern was present at later intervals (>288 hours post-dose) with a smaller inflection in magnitude (FIG. 10). The mean percent change in IOP from baseline also suggested similar patterns (FIG. 11).

Healon® treated eyes in Group 2 had mean IOP values approximately 10% lower than BSS treated eyes through 48 hours post-dose (FIG. 10). At 48 hours and beyond, the Healon® treated right eyes had mean IOP values lower than the BSS treated eyes, with the largest difference being early in the morning when the normotensive IOP values are typically the highest along the diurnal curve. As the mean IOP dropped during the day in the BSS treated eyes, the mean IOP values approached the mean IOP values for Healon® treated eyes. A minimal difference (<5%) between mean IOP values for the differently treated eyes at 288 hours post-dose and beyond (FIG. 10).

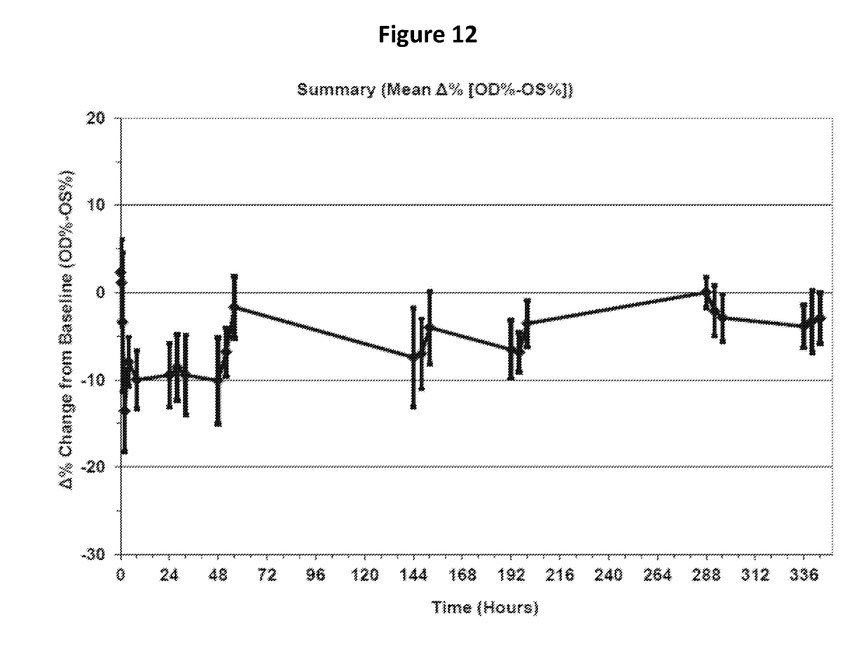

Based on the minimal IOP lowering effect of BSS injected into the SCS, the left eyes can be used to normalize the mean IOP values in the Healon® treated eyes. Upon normalization to baseline IOP values and in comparison to the contralateral eye, the effect of Healon® injected into the SCS can be seen to be the largest magnitude (−10%) mean IOP decrease through 48 hours post-dose (FIG. 12). A smaller magnitude of effect was observed between 48 and 200 hours postdose. As mentioned above, very little IOP lowering effect was observed beyond 288 hours post-dose.

Thus, SCS injections of BSS resulted in mean IOP values representative of diurnal variation. In contrast, SCS injections of Healon® resulted in a larger decrease HO %) in mean IOP values, which was most prominent through 48 hours postdose.

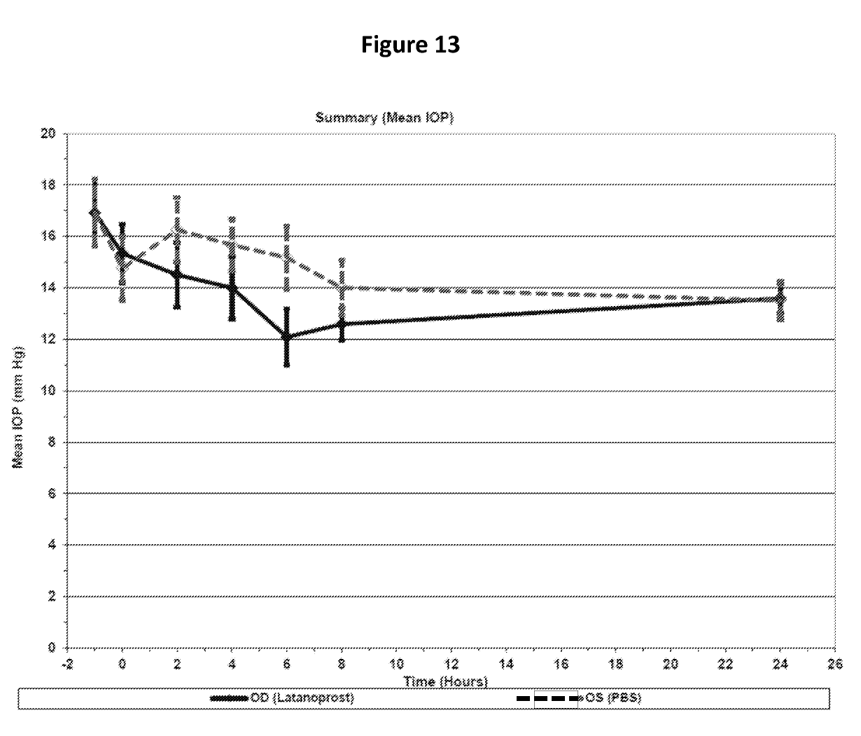

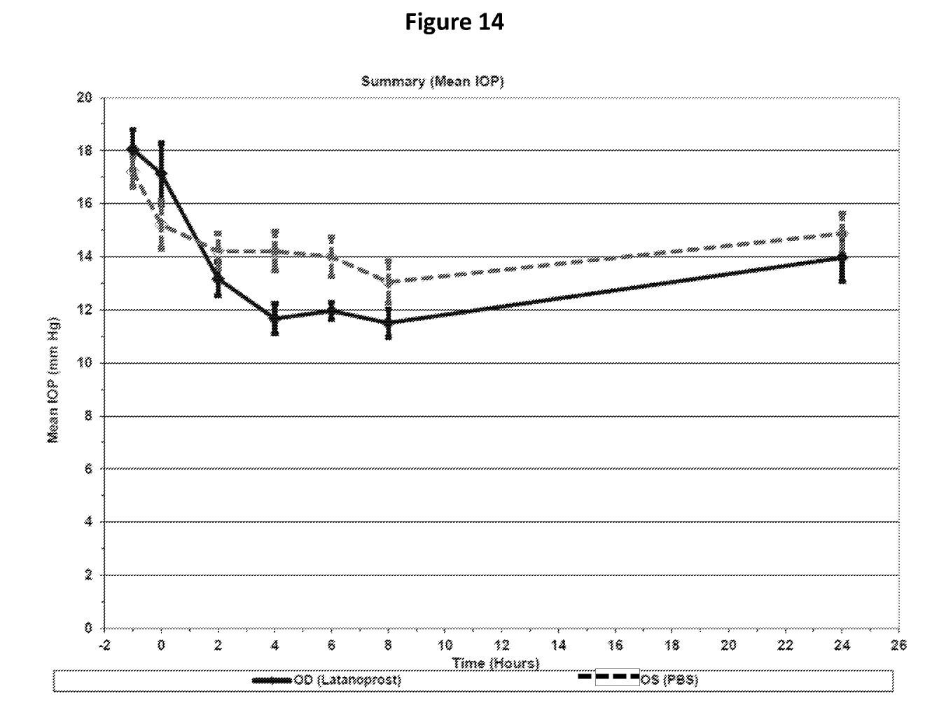

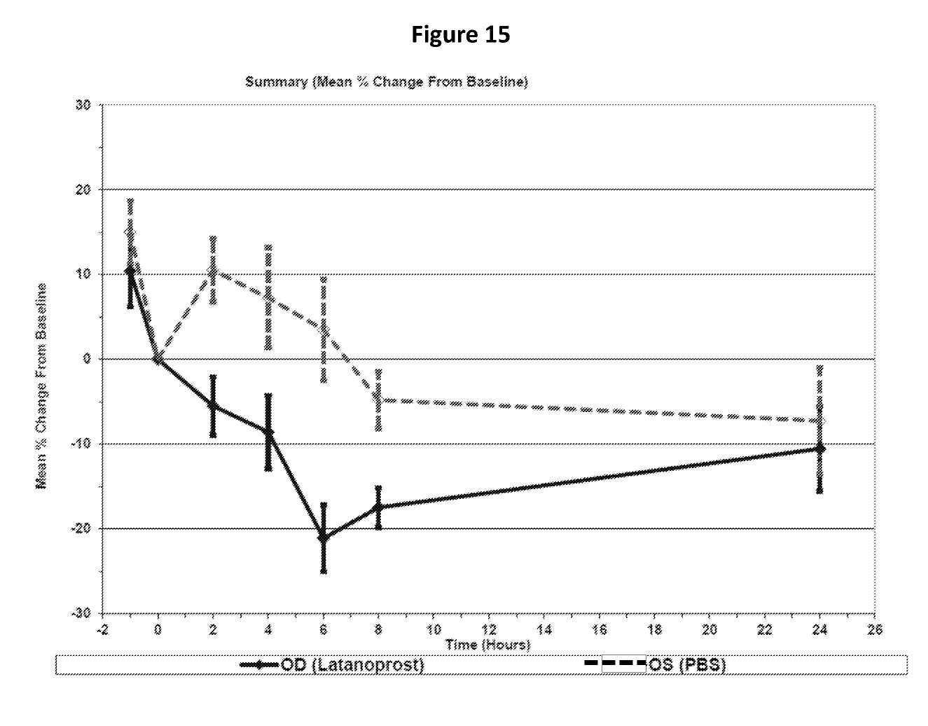

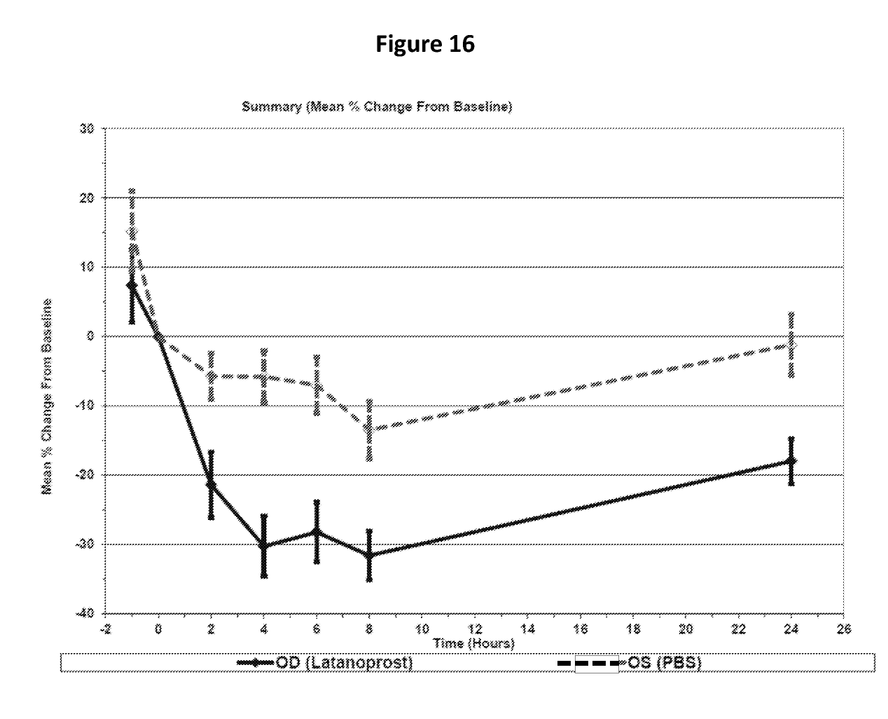

Following extended recovery after the SCS injections during the study phase, Groups 1 and 2 were administered a single topical ocular dose of latanoprost in the right eye and PBS in the left eye. Latanoprost decreased the IOP in the right eyes, while the IOP in the left eyes which received PBS were maintained at approximately baseline levels in accordance with diurnal variation (FIGS. 13-16). The mean maximum effective decrease (Emax) in IOP values of latanoprost dosed eyes was approximately 3-5 mm Hg, which was observed during the diurnal plateau between 4 and 8 hours postdose (Tmax). These data indicate the animals in both Groups 1 and 2 gave the typical response to latanoprost after the study phase which included anesthesia and suprachoroidal dosing.

In summary, the study indicated that suprachoroidal injection of a non-pharmacologically active injectable formulation provided a decrease in IOP in a non-obvious manner.

Publications, patents and patent applications cited herein are specifically incorporated by reference in their entireties for all purposes. While the described invention has been described with reference to the specific embodiments thereof it should be understood by those skilled in the art that various changes may be made and equivalents may be substituted without departing from the true spirit and scope of the invention.

We claim:

1. A method for reducing intraocular pressure (IOP) in a subject in need thereof comprising:
injecting a non-pharmacologically active formulation or placing a non-pharmacologically active solid implant into the suprachoroidal space (SCS) or the supraciliary space of the eye of the subject.

2. The method of claim 1, wherein the non-pharmacologically active formulation comprises a fluid injectate or a gel injectate.

3. The method of claim 2, wherein the fluid injectate increases aqueous outflow pathways of the eye.

4. The method of claim 3, wherein the aqueous outflow pathways of the eye are the trabecular meshwork (TM) outflow pathway and the uveoscleral outflow pathway.

5. The method of claim 2, wherein the injectate is water, an emulsion, or a hyaluronic acid based gel.

6. The method of claim 1, wherein the volume of the formulation is from about 10 μL to about 500 μL.

7. The method of claim 1, wherein the intraocular pressure (IOP) is reduced for at least about 12 hours, at least about 24 hours, at least about 2 days, at least about 3 days, at least about 4 days, at least about 5 days, at least about 6 days, at least about 1 week, at least about 2 weeks, at least about 3 weeks, at least about 1 month, at least about 2 months, at least about 3 months, at least about 4 months, at least about 5 months, or at least about 6 months following injection of the formulation or placement of the solid implant.

8. The method of claim 1, wherein the space between the anterior chamber and the SCS is expanded.

9. The method of claim 1, wherein the method improves drainage of the canal or uvea.

10. The method of claim 1, wherein the method reduces fluid production by the ciliary body (CB).

11. The method of claim 1, wherein the method increases outflow through the trabecular meshwork (TM) outflow pathway and/or increases outflow through the uveoscleral outflow pathway.

12. The method of claim 11, wherein the method increases outflow through both the TM and the uveoscleral outflow pathways.

13. The method of claim 11, wherein the method increases outflow by flushing the system and/or affecting the ciliary body (CB), and/or by causing ocular tissues to become more porous.

14. The method of claim 1, wherein the method causes mechanical deformation of the TM.

15. The method of claim 1, wherein the injectate creates an apparent an arc-shape in the SCS or the supraciliary space.

16. The method of claim 1, wherein the method comprises multiple injections of the formulation into the SCS or the supraciliary space, and wherein the multiple injections create an apparent arc-shape in the SCS or the supraciliary space.

17. The method of claim 1, further comprising an additional procedure for reducing IOP in the eye of the subject.

18. The method of claim 17, wherein the additional procedure is a surgery or the administration of a drug.

19. The method of claim 18, wherein the drug is administered to the subject via an ocular route of administration.

20. The method of claim 18, wherein the drug is present in a pharmacologically active injectable formulation.

21. The method of claim 18, wherein the drug is selected from the group consisting of cholinergic agents, latrunculins, ROCK inhibitors, prostaglandin analogues, α-adrenic receptor agonists, β-adrenergic receptor blockers, prostaglandin EP2 agonists, nitric oxid-donating prostaglandin F2α analogs, phosphylene iodide, and echothiopate iodide.

22. The method of claim 20, wherein the drug is selected from the group consisting of Dorzolamide/Timolol (Cospot), Carteolol, Bimatoprost (Lumigan), Latanoprost (Xalatan), Brimonidine, Betaxolol (Betoptic), Travoprost, Dorzolamide (Trusopt), Timolol (Betimol), Pilocarpine, Brinzolamide (Azopt), Iopidine, Alphagan P, Betagan, OptiPranolol, Istalol, Timoptic-XE, Neptazane, Diamox Sequels, Isopto Carpine, Isopto Carbachol, Pilopine HS gel, Pilocarpine CL ophthalmic solution USP, Combigan, Simbrinza suspension, Travatan Z, Lumigan, Zioptan, and Xalatan.

23. The method of claim 20, wherein the surgery is a minimally invasive glaucoma surgery (MIGS).

24. The method of claim 20, wherein the surgery comprises the application of a shunt.

25. The method of claim 20, wherein the method and the additional procedure provide an additive or synergistic reduction in IOP.

26. The method of claim 1, wherein the formulation is injected using a hollow microneedle.

27. The method of claim 1, wherein the subject is a mammal.

28. The method of claim 27, wherein the mammal is a human.

29. A method of treating glaucoma in a subject in need thereof comprising injecting a non-pharmacologically active formulation or placing a non-pharmacologically active solid implant into the suprachoroidal space (SCS) or the supraciliary space of the eye of the subject.

30. The method of claim 29, wherein the non-pharmacologically active formulation comprises a fluid injectate or a gel injectate.

31. The method of claim 30, wherein the fluid injectate increases aqueous outflow pathways of the eye.

32. The method of claim 31, wherein the aqueous outflow pathways of the eye are the trabecular meshwork (TM) outflow pathway and the uveoscleral outflow pathway.

33. The method of claim 31, wherein the injectate is water, an emulsion, or a hyaluronic acid based gel.

34. The method of claim 33, wherein the formulation has a volume of from about 10 μL to about 500 μL.

35. The method of claim 29, wherein the method reduces intraocular pressure (IOP) in the eye of the subject.

36. The method of claim 29, wherein the method improves drainage of the canal or uvea.

37. The method of claim 29, wherein the method reduces fluid production by the ciliary body (CB).

38. The method of claim 29, wherein the method increases outflow through the trabecular meshwork (TM) outflow pathway and/or increases outflow through the uveoscleral outflow pathway.

39. The method of claim 38, wherein the method increases outflow through both the TM and the uveoscleral outflow pathways.

40. The method of claim 38, wherein the method increases outflow by flushing the system and/or affecting the ciliary body (CB), and/or by causing ocular tissues to become more porous.

41. The method of claim 29, wherein the method causes mechanical deformation of the TM.

42. The method of claim 29, wherein the formulation or implant creates an apparent an arc-shape in the SCS or the supraciliary space.

43. The method of claim 29, wherein the method comprises multiple injections of the formulation into the SCS or the supraciliary space, and wherein the multiple injections create an apparent arc-shape in the SCS or the supraciliary space.

44. The method of claim 29, further comprising an additional procedure for reducing IOP in the eye of the subject.

45. The method of claim 44, wherein the additional procedure is a surgery or the administration of a drug.

46. The method of claim 45, wherein the drug is administered to the subject via an ocular route of administration.

47. The method of claim 45, wherein the drug is present in a pharmacologically active injectable formulation.

48. The method of claim 45, wherein the drug is selected from the group consisting of cholinergic agents, latrunculins, ROCK inhibitors, prostaglandin analogues, α-adrenic receptor agonists, β-adrenergic receptor blockers, prostaglandin EP2 agonists, nitric oxide-donating prostaglandin F2α analogs, phosphylene iodide, and echothiopate iodide.

49. The method of claim 45, wherein the drug is selected from the group consisting of Dorzolamide/Timolol (Cospot), Carteolol, Bimatoprost (Lumigan), Latanoprost (Xalatan), Brimonidine, Betaxolol (Betoptic), Travoprost, Dorzolamide (Trusopt), Timolol (Betimol), Pilocarpine, Brinzolamide (Azopt), Iopidine, Alphagan P, Betagan, OptiPranolol, Istalol, Timoptic-XE, Neptazane, Diamox Sequels, Isopto Carpine, Isopto Carbachol, Pilopine HS gel, Pilocarpine CL ophthalmic solution USP, Combigan, Simbrinza suspension, Travatan Z, Lumigan, Zioptan, and Xalatan.

50. The method of claim 45, wherein the surgery is a minimally invasive glaucoma surgery (MIGS).

51. The method of claim 45, wherein the surgery comprises the application of a shunt.

52. The method of claim 44, wherein the method and the additional procedure provide an additive or synergistic reduction in IOP.

53. The method of claim 29, wherein the formulation is injected using a hollow microneedle.

54. The method of claim 29, wherein the subject is a mammal.

55. The method of claim 54, wherein the mammal is a human.

56. A method of treating glaucoma in a human in need thereof comprising non-surgically administering a non-pharmacologically active injectable formulation to the anterior suprachoroidal space (SCS) or the supraciliary space of the eye of the human by using a hollow microneedle, whereupon administration, the intraocular pressure (IOP) is reduced.
